(19) United States
(12) Patent Application Publication
(10) Pub. No.: US 2003/0094175 A1
(43) Pub. Date: May 22, 2003

(54) Anti-hijacker system for airplane

(76) Inventor: Minh Hung Le, Montreal (CA)

Correspondence Address: MINH HUNG LE, 7443 BERRI St., MONTREAL, QC, H2R 2G6, CA

(21) Appl. No.: 09/992,697

(22) Filed: Nov. 16, 2001

(51) Int. Cl. A61M 16/00

(52) U.S. Cl. 128/203.12; 128/205.25

(57) ABSTRACT

The anti-hijack system proposed here can solve a critical point when hijackers just begin taking action onboard (airplane) or the pilots sense or receive a signal of hijack. This system peacefully resettles a situation without hurting onboard travelers, hijackers or even damaging an aircraft. That is a drug-induced sleep to be used and its system to be developed.

### SPECIFICATION

The proposed idea is to disable onboard hijackers' intention to hijack an airplane and to save all aboard as well as the aircraft and buildings from possible devastation as a result of the aircraft.

### BACK GROUND OF THE INVENTION

Hijacking an airplane occurs quite often in the world today. Marshals onboard were proposed and have been applied. I think this idea does not really bring a halt to hijacking. A reason being is the hijackers do not have respect even for their own lives. The exchange of gunfire can have serous consequences.

The free-boarding gate security check has its deficiencies i.e. employees having access to the plane for maintenance, cleaning and fueling etc. In addition, there is lax in passenger security check involving in transit passengers re-boarding.

The combination of the above two methods has not really appeased air voyagers' anxiety. Stray bullets could cause death or explosion in an aircraft. If the situation is not solved, the air business and other air-related industries will no doubt suffer severe economic loss.

As a solution, the use of drug-induced sleep Pentothal, Innoval, or Valium etc. could be introduced aboard the aircraft. These safe drugs could be used for all including children and infants. Additionally, they are inflammable and non-explosive [1]. With these advantages, the drug is appropriated to be used in aircrafts in case of urgency such as hijack. 1. Pages 31-32 chapter 8, the Medical Encyclopedia and Guide to Family Health Author Robert E. Rothenbeag M.D. F.A.C.S. Lexicon Publication, Inc. N.Y. 9^{th} edition 1991

### BRIEF SUMMARY OF THE INVENTION

How to peacefully subdue hijackers, without injury to all concerned or damage to the aircraft. The difficulties and inconveniences may be overcome by the use of drug-induced sleep and its anti-hijacker system. As soon as the pilots receive or sense a signal of hijack, they turn a valve to release the drug-induced sleep into the intended chambers and turn off another valve to stop supplying oxygen into masks. All onboard people (except pilots!!!) fall into an unconscious state. The pilots therefore have complete control of the situation.

### BRIEF DESCRIPTION OF THE SYSTEM

FIG. 1 The container with the two halves of main tube and the valve

FIG. 2 The anti-hijacker system

(a) The agent distributing system

(b) The observer system

FIG. 3 The system is installed inside the airplane

FIG. 4 The additional masks as the tools to distribute the drug-induced sleep

FIG. 5 The system along with the additional masks, monitor, and the electronic eyes inside the airplane.

### DETAIL DESCRPTION OF THE DRAWING

FIG. 1, the container mouth is covered with its cover. A hole is drilled on the container body and fixed with one end of the main tube. The main tube is cut into two parts. One end of the first half already is connected with the container while the other end is fixed with the valve side. The left valve side is fixed with one end of the second half main tube.

FIG. 2 (a), the free end of the second half main tube is branched with several long tubes on which many holes are drilled down. The diameter of the main tube is bigger than that of the long tube. The diameters of the holes become larger toward the end of the long tubes.

FIG. 2 (b) the electronics eyes are wired with the monitor located in the cockpit.

FIG. 3, the system is embedded inside an airplane. The container filled with the agent and the valves are located in the cockpit. The long tubes are installed on the airplane ceiling. The holes on the body of the long tubes are directing downward and horizontally

FIG. 4 the masks (already exist on the airplane) are also branched with the main tube to distribute the agent.

FIG. 5 the system and the masks are in the airplane. Additionally, a monitor is installed in the cockpit and some electronic eyes are located in the passengers' location and hung on the ceiling. The oxygen-supplying container already exists on the airplane

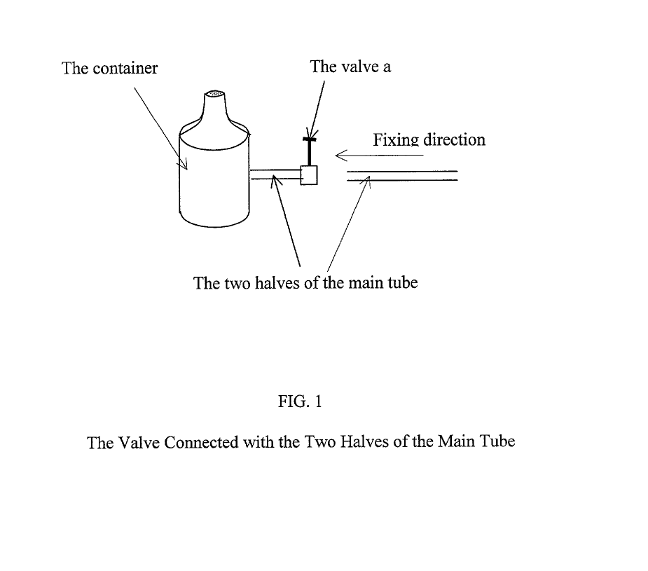

The embodiments of the invention in which an exclusive property is claimed are defined as follows:

1 In drawing that illustrates the body of the invention includes a tank filled with drug-induced sleep, a manual valve (or electronic panel), a main tube, and drilled-hole tubes.

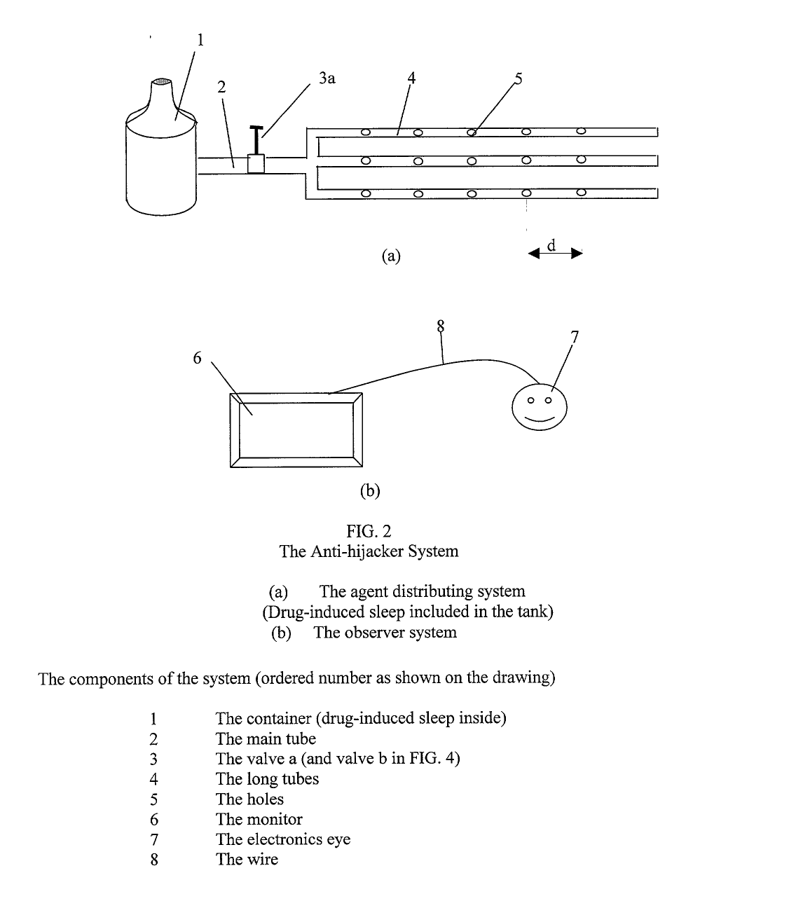

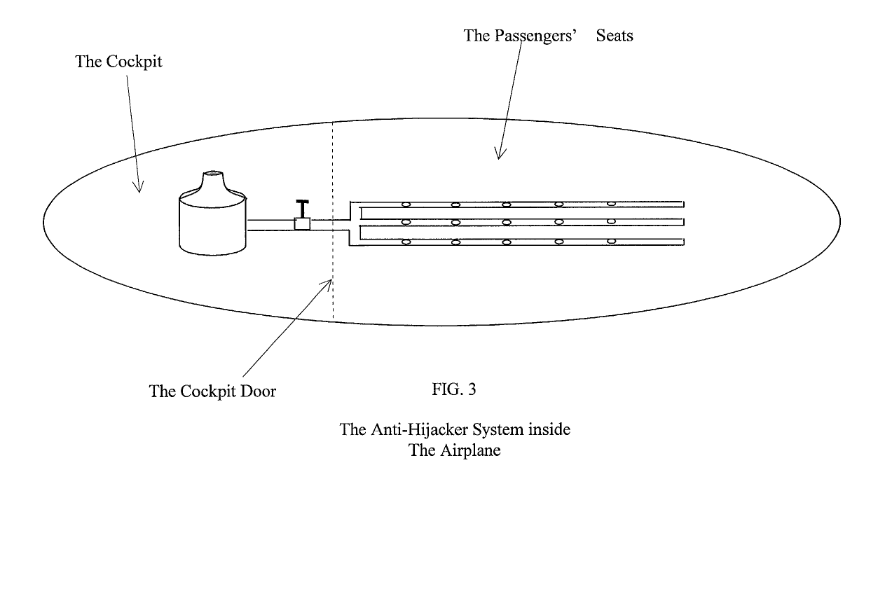

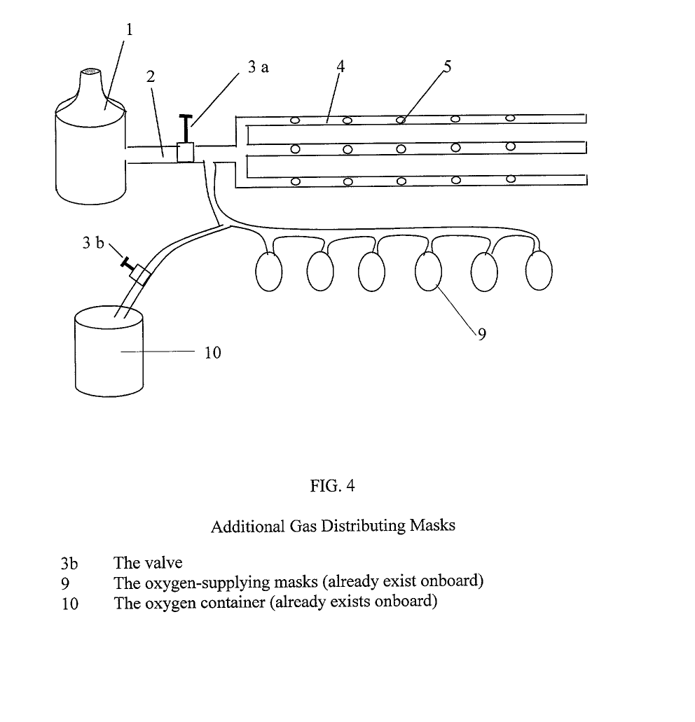

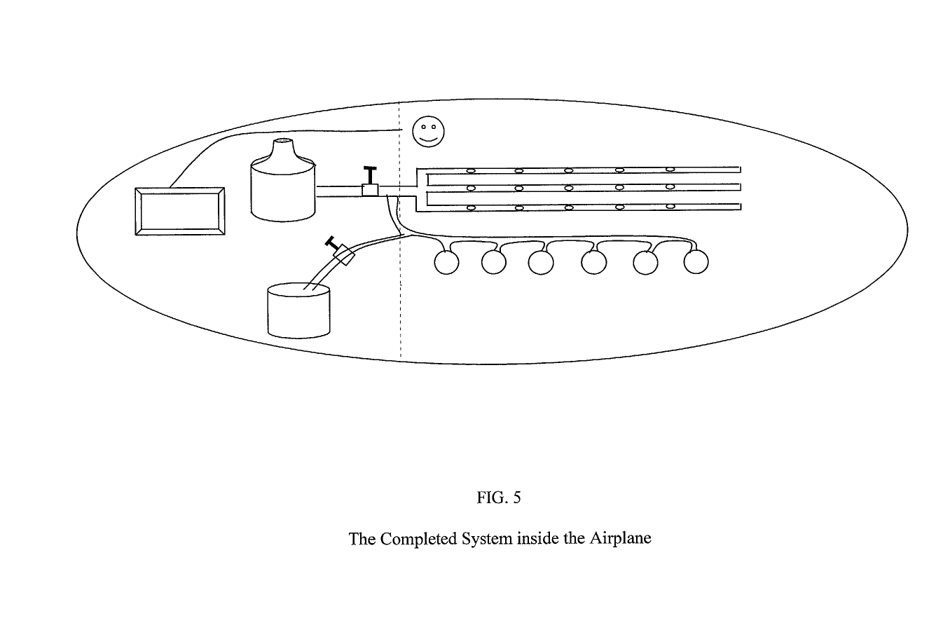

2 The agent inside the container 1 declared in claim 1 can be any kind of drug-induced sleep. However, this agent has to be approved by the anesthesiologists. The container 1 can be any size and shape. It is quite depending on the requirements such as the quantity of the agent to be released. The container is firmly covered at its mouth and its outlet (hole) is branched with a tube 2. A valve 3a is inserted in the main tube to control the agent flowing through the main tube 2 from the container 1 once the valve is in an open position. Several long tubes 4 are branched with the main tube 2 to bring the agent along the body and to the end of the airplane (as shown in FIG. 2). Several holes 5 with slightly different diameters are drilled down the long tubes 4. The separations d between the holes is calculated so that agent well distributes into the airplane through the holes 5. Note: the emergency-oxygen supplying masks 9 and the oxygen container already exist on board. They are branched with the drug-induced sleep container as shown in FIG. 4. The valve 3b should be inserted on the outlet of the oxygen container.

3 The electronic eyes 7 are in the passenger location. A wire 8 connects the electronics eyes 7 with the monitor 6 located in the cockpit.
